(19) United States
(12) Patent Application Publication
(10) Pub. No.: US 2026/0240493 A1
(43) Pub. Date: Aug. 20, 2026

(54) EXTERNAL STENT RESTENOSIS DEVICE

(71) Applicant: KONINKLIJKE PHILIPS N.V., EINDHOVEN (NL)

(72) Inventors: Bernardus Hendrikus Wilhelmus HENDRIKS, EINDHOVEN (NL); Gerardus Johannes Nicolaas DOODEMAN, VELDHOVEN (NL); Vipul Shrihari PAI RAIKAR, SOMERVILLE, MA (US); Leili SALEHI, WALTHAM, MA (US); Alyssa TORJESEN, EXETER, NH (US); René Leonardus Jacobus Marie UBACHS, EINDHOVEN (NL)

(21) Appl. No.: 19/470,948

(22) PCT Filed: Apr. 2, 2024
(86) PCT No.: PCT/EP2024/058902

§ 371 Date: Sep. 30, 2025

Related U.S. Application Data
(60) Provisional application No. 63/456,618, filed on Apr. 3, 2023.

(51) Int. Cl. A61B 5/00; A61B 5/02; A61B 5/05; A61B 5/07

(52) U.S. Cl. CPC A61B 5/4851; A61B 5/02007; A61B 5/05; A61B 5/076; A61B 5/7267

(57) ABSTRACT

A stent assessment system includes a transmit antenna configured to transmit an electromagnetic signal to an implanted stent; a receive antenna configured to receive a secondary electromagnetic signal produced by interaction between the electromagnetic signal and the implanted stent; and a hardware processor configured to determine an amount of restenosis on the implanted stent based on the secondary electromagnetic signal received by the receive antenna.

### FIELD

The following relates generally to the vascular stent arts, blood flow sensing arts, electromagnetic signal sensing arts, stenosis sensing arts, and related arts.

### BACKGROUND

When a coronary artery becomes (partially) obstructed a treatment may be required such as a balloon angioplasty followed by a stent placement to open up and restore the blood flow. A problem that may occur after placing a stent is in-stent restenosis, which is a situation where the metallic stent within the artery can cause inflammation leading to excess growth of blood vessel tissue in the stent causing a re-narrowing of the blood vessel. These in-stent restenosis may lead to late thrombosis and resultant heart attacks over time. Therefore, early detection of this in-stent restenosis is required (see, e.g., X. Chen, B. Assadsangabi, Y. Hsiang and K. Takahata, “Enabling angioplasty-ready “Smart” stents to detect in-stent restenosis and occlusion,” Adv. Sci. 5 (2018) p 1700560.).

Methods used to identify in-stent restenosis include duplex ultrasound and angiography. These methods are, however, difficult to apply over a longer time span. More recently, a method is proposed where the stent is made smart by equipping the stent with a microscale sensor (see, e.g., Chen et al.). A drawback of this method is that it requires a modification of the stent to include the sensor, and therefore cannot be applied to stents without this sensor.

A problem arises in how to detect a non-invasive in-stent restenosis without adding a sensor to the stent itself, or relying on an imaging device that may not be available in a doctor's office setting. Stent restenosis can occur after angioplasty and stent placement of an occlusion of the blood vessel. Since stent restenosis gradually occurs over time a method to detect restenosis in a non-invasive way is required that measures the restenosis growth over a longer period of time.

The following discloses certain improvements to overcome these problems and others.

### SUMMARY

In some embodiments disclosed herein, a system for stent assessment includes a transmit antenna configured to transmit an electromagnetic signal to an implanted stent, a receive antenna configured to receive a secondary electromagnetic signal produced by interaction between the electromagnetic signal and the implanted stent, and a hardware processor configured to determine an amount of restenosis on the implanted stent based on the secondary electromagnetic signal received by the receive antenna. In some embodiments, the hardware processor is configured to determine a resonance frequency of the implanted stent from the received secondary electromagnetic signal, for example using a frequency sweep, and determine the amount of restenosis based on the resonance frequency. In some embodiments, the amount of restenosis is determined by applying a machine learning (ML) model to the secondary electromagnetic signal received by the receive antenna. In some embodiments, the transmit antenna is a focusing antenna configured to focus the electromagnetic signal on the implanted stent. In some embodiments, the implanted stent includes an electrically conductive helix, and the transmit antenna is a loop antenna configured to electromagnetically couple with the electrically conductive helix of the implanted stent.

In some embodiments disclosed herein, the system set forth in the immediately preceding paragraph includes an implanted stent.

In some embodiments, disclosed herein, a stent assessment method includes: transmitting, via a transmit antenna, an electromagnetic signal to an implanted stent and receiving, via a receive antenna, a secondary electromagnetic signal produced by interaction between the electromagnetic signal and the implanted stent; and determining, via a hardware processor, an amount of restenosis on the implanted stent based on the secondary electromagnetic signal received by the antenna.

One advantage resides in detecting restenosis in a blood vessel. In some embodiments, the transmitting includes transmitting the electromagnetic signal with a magnetic field of the electromagnetic signal in a direction substantially parallel to a length direction of the stent. In some embodiments, the transmit antenna is configured to transmit the electromagnetic signal to the implanted stent, and the receive antenna is configured to receive the secondary electromagnetic signal produced by the interaction between the electromagnetic signal and the implanted stent. In some embodiments, the transmit antenna is secured to a person having the implanted stent with the transmit antenna oriented to transmit the magnetic field in a direction substantially parallel to a length direction of the stent.

Another advantage resides in non-invasively detecting an in-stent restenosis without altering an implanted stent in a patient.

Another advantage resides in early detection of disease progression and providing a treating physician with location specific information that is key to the planning of additional treatment.

Another advantage resides in monitoring and preventing restenosis at a stent implanted in a patient.

Another advantage resides in reducing or eliminating a need for imaging of a potential restenosis in a patient.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a stent apparatus in accordance with an embodiment of the present invention.

FIG. 2 diagrammatically illustrates a method of performing a stent assessment method using the apparatus of FIG. 1 in an embodiment of the present invention.

FIGS. 3 and 4 show other embodiments of the apparatus of FIG. 1.

FIG. 5 shows data generated by the apparatus of FIG. 1 in embodiments of the present invention.

### DETAILED DESCRIPTION

Approaches disclosed herein for monitoring stent restenosis are based on recognition that a conductive stent has an electrical resonance frequency which depends on the geometry (e.g. length) of the stent and the dielectric properties of its surrounding matter. When there is no restenosis in a blood vessel in which a stent is implanted, about half of the stent (e.g., the inside of the stent) is in contact with blood and the other half (e.g., the outside of the stent) with the blood vessel wall. When restenosis occurs in the blood vessel, a larger part of the stent will be covered with blood vessel wall cells rather than blood. Since the blood and the vessel wall have different dielectric properties, the electrical resonance frequency of the stent will change from the restenosis. Furthermore, the more blood vessel wall cells are present inside the stent, the more the dielectric properties will change and, therefore, also the electrical resonance frequency.

To detect this change in resonance frequency of the stent, a system is disclosed that comprises a transmit-receive antenna setup where, in some illustrative embodiments, the receive antenna detects a transmitted weak electrical field (E-field) generated by the interaction of the transmit antenna signal and the stent. A magnetic field (B-field) of the transmit antenna is significantly parallel to the length direction of the stent, so that the emitted weak E-field is substantially perpendicular to the incoming E-field limiting the interference of the two E-fields. A frequency sweep may be applied onto the transmit antenna to detect the resonance frequency. Using a look-up table (LOT), machine-learning (ML), or other process, the amount of restenosis can be determined by a hardware processor.

In some embodiments, X-ray imaging information or ultrasound imaging information (or any other suitable imaging modality information) is used to determine the orientation of the stent, such that the transmit antenna can be directed so that the transmit B-field is substantially parallel to the length direction of the stent. However, in many cases, the orientation of the stent respective to visible anatomy is known a priori, e.g., based on knowledge of where the stent was placed within the anatomy. In some cases, a pre-operative X-ray image acquired for use in planning the intravascular operation that placed the stent may be referenced, along with knowledge or annotation of the pre-operative image as to where the stent is located in the image. In another embodiment, the transmit antenna is designed such that the emitted E-field is focused on the stent to further improve a signal-to-noise ratio. In another embodiment, the transmit and receive antenna is in the form of a patch that can be placed on the chest of the patient.

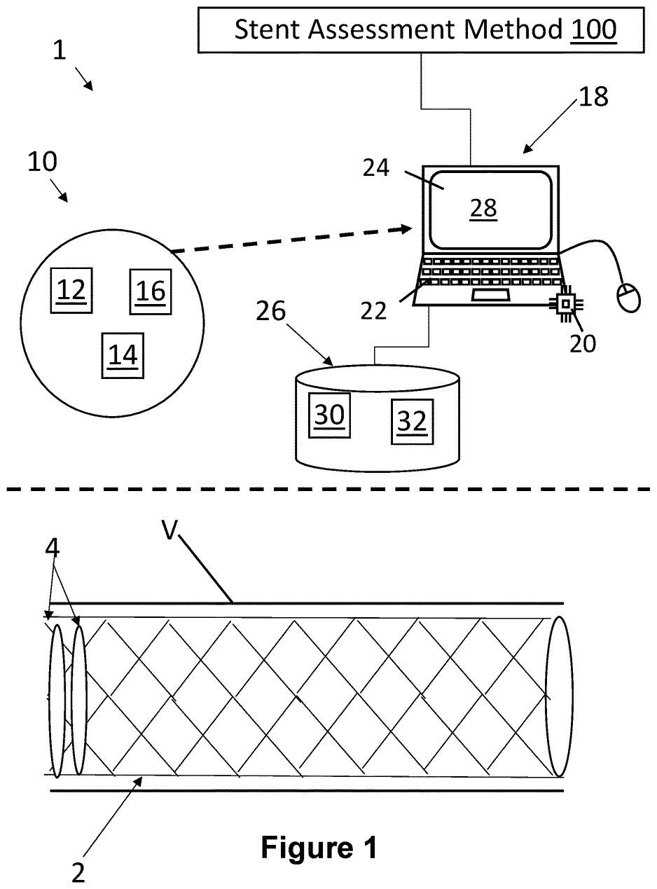

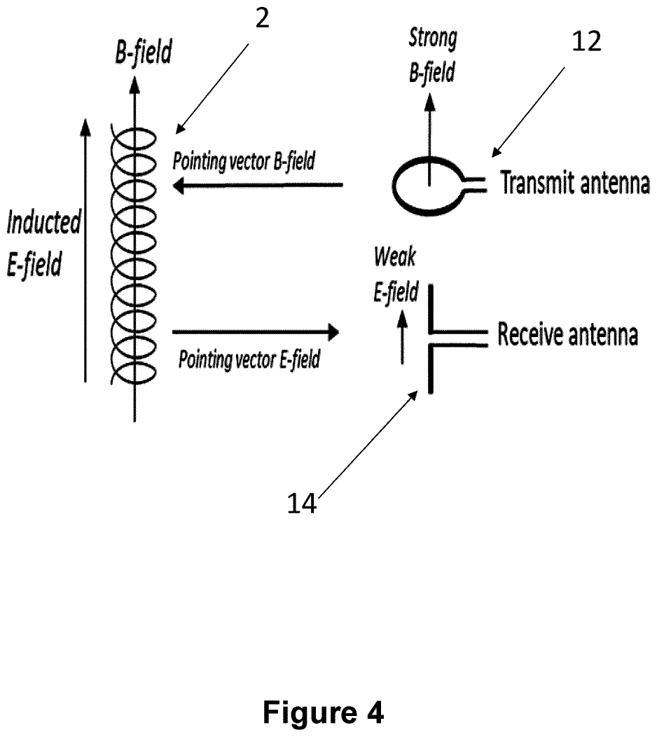

With reference to FIG. 1, an illustrative stent apparatus (system) 1 is diagrammatically shown, which includes hardware and software for performing the stent restenosis monitoring in embodiments of the present invention. As shown in FIG. 1, the apparatus 1 includes a vascular therapy device 2 (e.g., a self-expanding stent which can be self-expanding, a self-expanding filter, and so forth) that is implanted into a blood vessel V. The stent may be located substantially anywhere in the patient's body, depending on the treatment being provided. For example, the stent 2 may be disposed in a cardiac blood vessel, a pulmonary blood vessel, a peripheral blood vessel in an arm or leg, or so forth. In some embodiments, the stent 2 comprises a self-expanding Nitinol stent. Although not shown in FIG. 1, in some embodiments the stent 2 may include an electrically conductive helix (see FIG. 4).

Delivery of the stent 2 into the blood vessel V may be accomplished, for example, using a catheter or other intravascular instrument (not shown) that is inserted into a blood vessel via an incision and moved to the treatment site where the stent 2 is deployed (implanted). Various known stent delivery instrument systems can be used for this purpose. After deployment of the stent 2 into the blood vessel V, the intravascular instrument is withdrawn, leaving the deployed stent in place. In the case of a self-expanding (e.g., nitinol) stent, the stent delivery typically entails compressing the stent into a recess at or near the tip of the intravascular instrument, and releasing the stent by pushing it out of the recess using a suitable mechanism (e.g., wire-driven or cable-driven) of the intravascular instrument. In another embodiment, the stent (which in this approach may or may not be self-expanding) is deployed and an inflatable balloon at or near the tip of the interventional instrument is positioned inside the deployed stent and inflated to expand and press the stent into the wall of the blood vessel V. Typically, the stent 2 is a hollow tube which, thus, serves to increase the blood vessel diameter to relieve the stenosis. As further shown in FIG. 1, in some embodiments, the stent 2 may optionally include one or more radiopaque markers 4 (two of which are shown in FIG. 1, although any suitable number of markers can be used), to facilitate visualization of the stent during the stent implantation process.

The apparatus 1 also includes a stent assessment system 10 configured to determine an amount of restenosis on the stent 2 in the blood vessel V. As shown in FIG. 1, the stent assessment system 10 includes a transmit antenna 12 configured to transmit an electromagnetic signal to the implanted stent 2. In some embodiments, the transmit antenna 12 may be transmitted to the implanted stent 2 by the electromagnetic signal coupling with the electrically conductive helix of the implanted stent 2. In one example embodiment, the transmit antenna 12 is a focusing antenna configured to focus the electromagnetic signal on the implanted stent 2. In another embodiment, the transmit antenna 12 is a dipole antenna. In a further embodiments, the transmit antenna 12 is a loop antenna configured to electromagnetically couple with the electrically conductive helix of the implanted stent 2.

The stent assessment system 10 also includes a receive antenna 14 that is configured to receive a secondary electromagnetic signal produced by interaction between the electromagnetic signal and the implanted stent 2. In one embodiment, the receive antenna 14 receives the secondary electromagnetic signal by the coupling of the electromagnetic signal with the electrically conductive helix of the implanted stent 2. The receive antenna 14 may be configured to be secured to the person oriented substantially perpendicularly to the transmit antenna 12. In some embodiments, the receive antenna 14 can be a dipole antenna.

The stent assessment system 10 also includes a hardware processor 16 configured to determine an amount of restenosis on the implanted stent 2 based on the secondary electromagnetic signal received by the receive antenna 14. In some embodiments, the hardware processor 16 is configured to determine a resonance frequency of the implanted stent 2 from the received secondary electromagnetic signal, and determine the amount of restenosis based on the resonance frequency. To do so, in some embodiments, the transmit antenna 12 is be configured to sweep a frequency of the electromagnetic signal, and the hardware processor 16 is configured to determine the resonance frequency of the implanted stent 2 based on the received secondary electromagnetic signal during the sweep. In another embodiment, the hardware processor 16 comprises an antenna configured to transmit data from the transmit antenna 12 and the receive antenna 14 to an electronic processing device 18 for extracting the resonance frequency (or other parameters) based on the received secondary electromagnetic signal. In some embodiments, the stent assessment system 10 (transmit antenna 12, receive antenna 14, and hardware processor 16) is configured as a device that attaches to skin of the patient (as shown in FIG. 1). In other embodiments, the transmit antenna 12, receive antenna 14, and hardware processor 16 may be configured at different locations on or off the patient.

In some embodiments, the transmit antenna 12, the receive antenna 14, and/or the hardware processor 16 can be disposed on a patch that is secured to the patient (diagrammatically shown in FIG. 1 with elements 12, 14, 16 in a circle). In addition, in some embodiments, the stent assessment system 10 is secured outside of a portion of the patient, and the stent 2 is disposed in the blood vessel V of the patient (diagrammatically represented in FIG. 1 via a dashed line).

FIG. 1 further shows the electronic processing device 18, such as a workstation computer, or more generally a computer. The electronic processing device 18 may additionally or alternatively include a server computer or a plurality of server computers, e.g., interconnected to form a server cluster, cloud computing resource, or so forth, to perform more complex computational tasks, such as estimating the restenosis from the signals received from the receive antenna 14. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The hardware processor 16 of the stent assessment system 10 can be in electronic communication with the electronic processing device 18 (i.e., via the Internet). In some embodiments, the hardware processor 16 can be omitted, and the electronic processing device 18 performs the operations to determine the amount of restenosis. (Viewed alternatively, the hardware processor 16 and the electronic processing device 18 can be combined as a single processor).

In some embodiments, the non-transitory storage media 26 may include a look-up table 30 storing values of blood and blood vessel properties of the patient, and values of properties of the implanted stent 2. In another approach, a look-up table or empirical equation can be stored which maps resonance frequency to an amount of restenosis. This look-up table or empirical equation may be derived from experiments in which, for example, ground truth restenosis for some calibration examples is determined by an approach such as duplex ultrasound and angiography, and the secondary electromagnetic signal received by the receive antenna 14 for those calibration examples is also measured using the antennae 12 and 14 to provide the mapping 30 from measured resonance frequency to amount of restenosis. In another calibration approach, bioelectrical modeling of the stent 2 with varying amounts of restenosis can be used as the calibration examples, where the resonance frequency is modeled using Maxwell's equations and stored values of blood and blood vessel dielectric properties. In clinical use, the electronic processing device 18 is configured to determine the amount of restenosis by inputting the determined electrical resonance frequency into the look-up table or empirical equation 30, and outputting a value indicative of the amount of restenosis from the look-up table 30. In other embodiments, the non-transitory storage media 26 can store a machine-learning (ML) model 32 (e.g., an artificial neural network (ANN), a support vector machine (SVM), and so forth), suitably trained on actual or virtual (i.e., modeled) calibration examples, such as those just described. The electronic processing device 18 is configured in these embodiments to determine the amount of restenosis by applying the trained ML model 32 to the secondary electromagnetic signal received by the receive antenna 14. The electronic processing device 18 can then output indication of the determined amount of restenosis (e.g. a numerical value, a percentage, and so forth) on the GUI 28 of the display device 24.

The hardware processor 16 and/or the electronic processing device 18 is configured as described above to perform a stent assessment method or process 100. The non-transitory storage medium 26 stores instructions which are readable and executable by the at least one electronic processor 20 to perform disclosed operations including performing the vascular therapy method or process 100. In some examples, the method 100 may be performed at least in part by cloud processing.

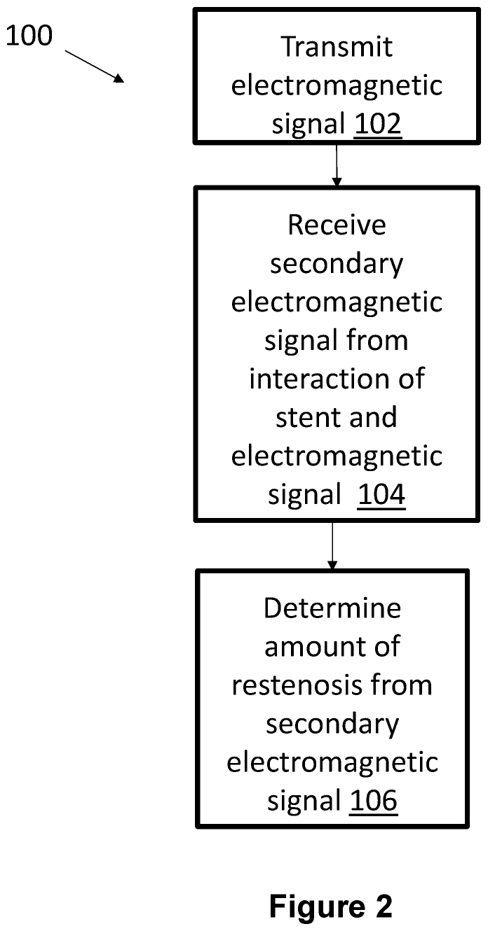

Referring to FIG. 2, and with continuing reference to FIG. 1, an illustrative embodiment of the stent assessment method 100 is diagrammatically shown as a flowchart. To begin the method 100, the stent 2 is deployed into the blood vessel V at a treatment location in a blood vessel V of a patient. The stent assessment system 10 is configured which, in some embodiments, may include attachment to skin of the patient.

At an operation 102, an electromagnetic signal is transmitted, using the transmit antenna 12, to the implanted stent 2. To do so, the electromagnetic signal is transmitted so that a magnetic field of the electromagnetic signal is oriented in a direction substantially parallel to a length direction of the stent 2. In some embodiments, the transmit antenna 12 is secured to the patient to be oriented to transmit the magnetic field in a direction substantially parallel to a length direction of the stent 2.

At an operation 104, the secondary electromagnetic signal produced by interaction between the electromagnetic signal and the implanted stent 2 is received by the receive antenna 14. The secondary electromagnetic signal is transmitted to the hardware processor 16 and/or the electronic processing device 18.

At an operation 106, the hardware processor 16 (and/or the electronic processing device 18) determines an amount of restenosis on the implanted stent 2 based on the secondary electromagnetic signal. To do so, in some embodiments, the transmit antenna 12 is configured to sweep a frequency of the electromagnetic signal and the hardware processor 16 is configured to determine the resonance frequency of the implanted stent 2 based on the received secondary electromagnetic signal during the sweep. In some embodiments, the electronic processing device 18 is configured to apply the ML model 32 to the secondary electromagnetic signal to determine the resonance frequency of the implanted stent 2. In some embodiments, the electronic processing device 18 is configured to input the secondary electromagnetic signal to the look-up table 30 to determine the resonance frequency of the implanted stent 2.

Advantageously, the method 100 can be performed in a doctor's office setting without requiring medical imaging equipment. For example, in one contemplated implementation the antennae 12 and 14 are disposed on a band, adhesive patch, or the like that can be placed onto the patient by the doctor to perform the measurement. While operation 106 is described for a single session, it is also noted that in some embodiments the trend of the secondary electromagnetic signal measured by the receive antenna 14 from one doctor's visit to the next is recorded to produce a trendline over several weeks or months or longer. If this trendline is flat (little or no change in resonance frequency over time) this is a good indication that restenosis is not occurring; whereas, if this trendline shows a significant shift in the resonance frequency over time this suggests significant restenosis may be occurring, which could then provide a basis on which the patient's physician might order duplex ultrasound or angiography to confirm and more quantitatively assess the suspected restenosis.

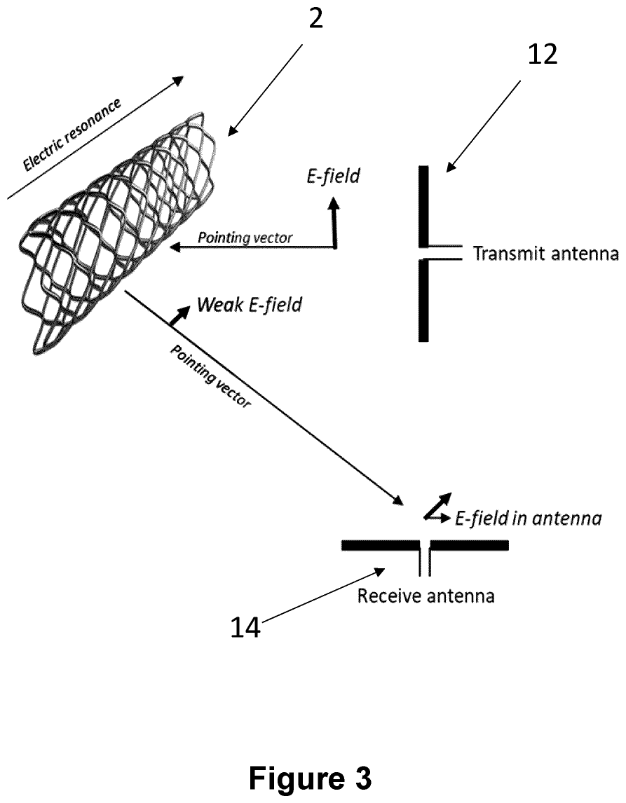

Referring now to FIG. 3, an illustrative example of one embodiment of the stent apparatus 1 and the method 100 is described. FIG. 3 shows an example arrangement of the stent 2, the transmit antenna 12, and the receive antenna 14. The resonance frequency of the stent 2 is •=••(2•••••••••.) with • being the speed of light (3×10⁸ m/s) and •. being the relative permittivity of the surrounding matter. The small size of the stent 2 leads to very high resonant frequencies, surrounded with blood (•. •81) and 2 cm length, the resonance frequency is about 92 MHz. Surrounded with fat (•. •13) the frequency raises to about 580 MHz. It is possible to detect the resonance frequency by applying an electromagnetic wave with a pointing vector with a certain angle with respect to the length axis of the stent 2.

The stent 2 can transmit a secondary electromagnetic wave with an electric field parallel to the length axis of the stent 2, which can be received by the receive antenna 14 which is oriented perpendicular to the transmit antenna 12 and placed in a position where the reception of the transmitted E-field is at a minimum.

The resonance frequency •=••(2•••••••••.) shifts if permittivity properties of matter (i.e., blood, a wall of the blood vessel V, and so forth) around the stent 2 changes. The resonance frequency and damping caused by surrounding matter can be detected by applying a frequency swept transmitting signal and analyzing the strength and phase of the received signal over frequency. A disadvantage of high frequencies is the absorption of the electromagnetic wave in the human body, but with small bandwidth reception and a slow frequency scan, the frequency response signal can be found.

In an alternative embodiment, as shown in FIG. 4, the stent 2 can comprise a conductive spiral stent 2 which converts the magnetic field of an electromagnetic wave into an electric field. The spiral acts as a coil inductance with capacitance between the coil windings. The resonance frequency •=1•(2•••̅•̅). The inductance increases with an increase of permeability of the surrounding matter, and the capacitance increases linear with the permittivity of the surrounding matter. In this embodiment, the conductive spiral could form the entirety of the stent, or the stent could include a polymer or other electrically non-conductive base structure for the stent.

If well aligned, the generated electric field vector orientation is parallel to the received magnetic field vector. This means the generated electromagnetic field polarization is perpendicular to the transmitted field. The transmit antenna 12 may be a magnetic loop antenna, transmitting a strong magnetic field and weak electric field, while the receive antenna 14 can be a dipole antenna which is primarily sensitive for electric fields. The suppression of the transmitted signal in the receive antenna 14 depends on the positioning of both antennas with respect to each other. Optimal alignment is required.

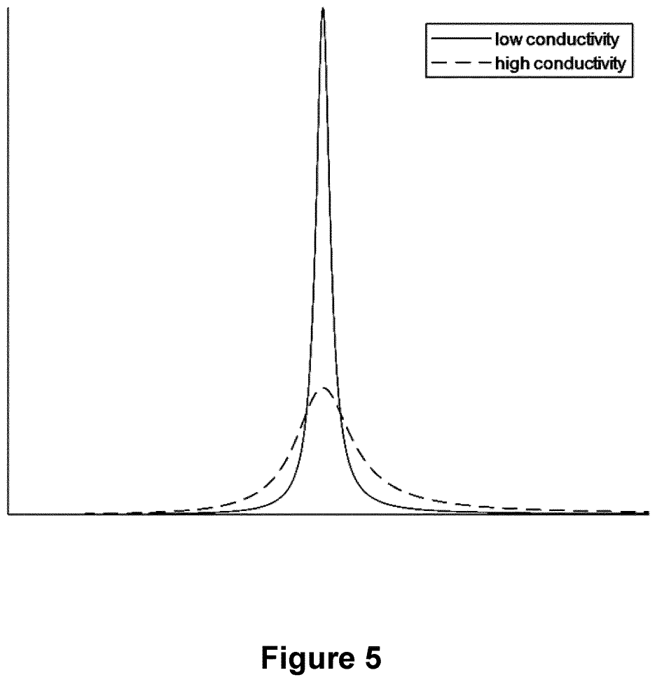

The conductivity of the surrounding matter affects the amplitude over frequency response of the stent 2. Increased conductivity leads to a wider bandwidth of the response signal. FIG. 5 shows an example data curve, in which the shape of the curve is a measure for the conductivity of the surrounding matter. The frequency response curve can be determined by a frequency sweep. An alternative method in other embodiments is to use an amplitude modulated carrier, where the modulation is a series of sync pulses with a defined frequency bandwidth. The received signal contains amplitude and phase over this bandwidth and the repeated series of received pulses contain the frequency response of the stent 2.

In some embodiments, the transmit antenna 12 can be designed to focus the transmitted beam. For example, a suitable focusing transmit antenna is disclosed in S. Kim, J. S. Ho and A. S. Y. Poon, “Midfield wireless powering of subwavelength autonomous devices”, Phys. Rev. Lett. 110 (2013), page 203905.

Dielectric tissue properties are of blood and blood vessel walls are known (see, e.g., S. Gabriel, R. W. Lau and C. Gabriel, “The dielectric properties of biological tissues: III. Parametric models for dielectric spectrum of tissues”, Phys. Med. Biol. 41 (1996) p. 2271-2293). Table 1 shows a list of permittivity and electrical conductivity for blood and blood vessel wall.

TABLE 1

| | | Blood | | Blood vessel wall | |
|---|---|---|---|---|---|
| | | | Elec. Cond. | | Elec. Cond. |
| Frequency | | Permittivity | (S/m) | Permittivity | (S/m) |
| 100 | Hz | 5260.0 | 0.700 | 5090000.0 | 0.278 |
| 1 | kHz | 5260.0 | 0.700 | 149000.0 | 0.307 |
| 10 | kHz | 5250.0 | 0.700 | 7690.0 | 0.313 |
| 100 | kHz | 5120.0 | 0.703 | 930.0 | 0.319 |
| 1 | MHz | 3030.0 | 0.822 | 218.0 | 0.327 |
| 10 | MHz | 280.0 | 1.10 | 109.0 | 0.345 |
| 100 | MHz | 76.8 | 1.23 | 59.8 | 0.462 |
| 1 | GHz | 61.1 | 1.58 | 44.6 | 0.729 |

In Table 1, a list of the dielectric properties of blood and blood vessel wall is given for various frequencies of the electromagnetic field. The results show that the dielectric properties of blood and blood vessel wall are different for all frequencies. As a result, the resonance frequency of the stent will alter when restenosis occurs since more parts of the stent 2 will be covered with blood vessel wall tissue instead of blood. Example calculations of the resonance frequency based on permittivity, electrical conductivity, etc. for blood and blood vessel wall used in embodiments of the present invention are described above with respect to FIGS. 3 and 4.

In some embodiments, the antennas 12, 14 are preferably made of a flexible material like a metal foil such that this can be integrated into a body patch. The required electronics and power source to drive the transmit and receiving antenna 12, 14 can be a separate device connected to the patch via cables or can be directly integrated into the patch. The patch is attached to the chest of the patient near the heart for optimal signal transfer.

Known signal processing methods when applied to tissue characterization can potentially be susceptible to noisy readings which can in turn lead to imprecise measurements of material properties of the tissue being probed. In addition, when a stent is implanted, it is expected to be covered by tissue as a natural biochemical response to the presence of a foreign body. However, excessive tissue growth that narrows the lumen is the cause of concern and may be subtle. This makes creating a decision surface for classification challenging. One way to tackle this problem, in embodiments, is to utilize machine learning and data driven approaches that are sensitive to such subtle signal changes. A straightforward way of generating a decision surface, in embodiments, is to classify the time-series RF signal (e.g., the entire spectrum shown in FIG. 5) using a supervised classifier such as a support vector machine (SVM) as the ML component 32. These processes may also be performed, in embodiments, by detecting and classifying time-frequency spectrograms using convolutional neural network architectures. In embodiments, a single shot detector such as YOLO can be used to detect and classify the spectrogram to its corresponding label of the level of ISR. In addition, in embodiments, multifactorial input that also includes information from other patient metrics can be used to create a robust model.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system for stent assessment, the system comprising:
a transmit antenna configured to transmit an electromagnetic signal to an implanted stent;
a receive antenna configured to receive a secondary electromagnetic signal produced by interaction between the electromagnetic signal and the implanted stent; and
a hardware processor configured to determine an amount of restenosis on the implanted stent based on the secondary electromagnetic signal received by the receive antenna.

2. The system of claim 1, wherein the hardware processor is further configured to:
determine a resonance frequency of the implanted stent from the received secondary electromagnetic signal; and
determine the amount of restenosis based on the resonance frequency.

3. The system of claim 2, wherein the transmit antenna is configured to sweep a frequency of the electromagnetic signal; and the hardware processor is configured to determine the resonance frequency of the implanted stent based on the received secondary electromagnetic signal during the sweep.

4. The system of claim 2, wherein the receive antenna is configured to be secured to a person oriented substantially perpendicularly to the transmit antenna.

5. The system of claim 3, wherein the transmit antenna is configured to be secured to a person by a patch on which or within which the transmit antenna is disposed.

6. The system of claim 1, wherein the hardware processor is further configured to:
input the determined electrical resonance frequency into a look-up table; and
output a value indicative of the amount of restenosis from the look-up table.

7. The system of claim 1, wherein the hardware processor is further configured apply a machine-learning model to the secondary electromagnetic signal received by the receive antenna to determine the amount of restenosis.

8. The system of claim 1, wherein the transmit antenna is a focusing antenna configured to focus the electromagnetic signal on the implanted stent.

9. The system of claim 1, wherein the transmit antenna is a dipole antenna.

10. The system of claim 1, wherein the receive antenna is a dipole antenna.

11. The system of claim 1, wherein the implanted stent includes an electrically conductive helix and the transmit antenna is a loop antenna configured to electromagnetically couple with the electrically conductive helix of the implanted stent.

12. The system of claim 1, wherein the hardware processor is further configured to:
output an indication of the determined amount of restenosis.

13. The system of claim 1, further comprising:
the implanted stent.

14. A method for assessing a stent, the method comprising:
transmitting, via a transmit antenna, an electromagnetic signal to an implanted stent;
receiving, via a receive antenna, a secondary electromagnetic signal produced by interaction between the electromagnetic signal and the implanted stent; and
determining, via a hardware processor, an amount of restenosis on the implanted stent based on the received secondary electromagnetic signal.

15. The method of claim 14, wherein the transmitting includes transmitting the electromagnetic signal with a magnetic field of the electromagnetic signal in a direction substantially parallel to a length direction of the stent.

16. The method of claim 15, further including:
securing the transmit antenna to a person having the implanted stent with the transmit antenna oriented to transmit the magnetic field in a direction substantially parallel to a length direction of the stent.

17. The method of claim 16, further comprising:
sweeping, via the transmit antenna, a frequency of the electromagnetic signal; and
determining, via the hardware processor, the resonance frequency of the implanted stent based on the received secondary electromagnetic signal during the sweep.

18. The method of claim 16, further including:
disposing the transmit antenna and the receive antenna on a portion of the patient, and disposing the stent inside a blood vessel of the patient.

19. The method of claim 14, further comprising:
determining, via the hardware processor, the amount of restenosis by applying a machine-learning model to the secondary electromagnetic signal received by the at least one antenna.

20. A non-transitory computer-readable storage medium having stored a computer program comprising instructions, which, when executed by a processor, cause the processor to:
obtain a secondary electromagnetic signal produced by interaction between an electromagnetic signal and an implanted stent, wherein the electromagnetic signal is transmitted to the implant stent by a transmit antenna and the secondary electromagnetic signal is received by a receive antenna; and
determine an amount of restenosis on the implanted stent based on the received secondary electromagnetic signal.
